(19) United States
(12) Patent Application Publication
(10) Pub. No.: US 2017/0299579 A1
(43) Pub. Date: Oct. 19, 2017

(54) VITELLINE MEMBRANE AS A MODEL OF THE BLOOD BRAIN BARRIER

(71) Applicant: Joel Steven Goldberg, Hillsborough, NC (US)

(72) Inventor: Joel Steven Goldberg, Hillsborough, NC (US)

(21) Appl. No.: 15/337,155

(22) Filed: Oct. 28, 2016

(51) Int. Cl. G01N 33/50; G01N 33/483; G01N 33/94

(52) U.S. Cl. CPC G01N 33/5088; G01N 33/94; G01N 33/4833; G01N 2333/465

(57) ABSTRACT

The vitelline membrane of an egg is a novel, inexpensive and useful model of the human blood brain barrier (BBB). FD&C Blue #1, FD&C Red #40, FD&C Red #3, quinine and fluorescein do not cross the vitelline membrane and do not cross the human BBB. Ethanol, diethyl ether, 2-chloroprocaine and acetaminophen, cross the vitelline membrane and are known to cross the human BBB. Using the vitelline membrane, as model of the human BBB, may decrease the time and cost of discovering lead drugs for the treatment of central nervous system diseases.

### CROSS-REFERENCES TO RELATED APPLICATIONS

None

### FEDERALLY FUNDED RESEARCH

Not applicable

### BACKGROUND OF THE INVENTION

The blood brain barrier (BBB) is a relatively impermeable barrier to drugs. This protective barrier makes it difficult to design medications that can treat diseases of the central nervous system (CNS). Making the development of drugs that cross the BBB even more challenging, the properties of the BBB in humans may not be the same as in other mammals.

The BBB is formed by capillary epithelial cells which contact with pericytes and astrocytes as well as brain endothelial cells which form a continuous line of tight and adherens junctions. (Wilhelm & Krizbai, 2014) This combination of epithelial and endothelial cells forms a membrane that is very exclusive. Although active transport mechanisms can influx and efflux substances across the BBB most lipophilic drugs cross the barrier by diffusion.

There are a number of models and criteria that are used to predict whether a compound will cross the BBB but there are no in vitro models to accurately determine whether a compound will cross the human BBB. Models are developed empirically. Some methods to estimate a compound's ability to permeate the BBB include: (Naik & Cucullo, 2012; Wilhelm & Krizbai, 2014)
1. Lipinski's 4 criteria including: 1) Log p, 2) Molecular weight, 3) Number of hydrogen bond donors and 4) Number of hydrogen bond acceptors. (Pajouhesh & Lenz, 2005)
2. Permeability of Caco-2 cells that use the cell line of human epithelial colorectal adenocarcinoma cells as an approximation of the BBB.
3. PAMP (partial artificial membrane permeability) testing of lead drugs that use lipid impregnated membranes interspaced between a donor and acceptor compartment.
4. Computer-assisted structure-based design known also as in silico models use computational information such as solubility, molecular size, charge, hydrogen bonding and lipophilicity to predict BBB permeability.
5. Initially developed for drug purification, immobilized artificial membrane (IAM) chromatography which is comprised of a lipid monolayer constructed on a chromatographic material has also been used to predict BBB permeability.

Each of these methods have shortcomings for determining if a compound will cross the human BBB. Administration to human volunteers is the best test to determine if compounds will cross the human BBB. However, administration of potential medications to humans is not without risks and requires more intensive and extensive animal trials prior to human administration. In fact, developing a new medication to market that treats diseases of the CNS is on average more costly and time consuming (12-16 years vs. 10-12 years) compared to developing a medication that treats other diseases that do not affect the CNS. (Wilhelm & Krizbai, 2014)

Aside from previously described criteria and methods to estimate whether a compound will cross the human BBB, “common sense” pharmacology requires that the metabolites of a prodrug be “familiar” or have a known presence in the CNS. (Goldberg, 2010) Lipophilic examples of such familiar conjugates of active drugs that can form ester prodrugs include cholesterol and selected fatty acids such as palmitic and linoleic acids. (Goldberg, 2011)

Unlike the BBB in living organisms, the vitelline membrane, the membrane that encloses the yolk in an egg, is not comprised of cells. (FIG. 1) The vitelline membrane is comprised of fibrils and amorphous substances in layers such that there is an outer and inner layer. (Bellairs, 1963) There are only a few citations that discuss the permeability of the vitelline membrane mostly describing the permeability to amino acids and macromolecules. (Garcia, Pons, Alemany, & Palou, 1983; Pons, 1985) No literature compares the vitelline membrane to the human BBB.

This invention is a new model of the BBB using the vitelline membrane of an egg to predict whether a compound may cross the human BBB.

### DESCRIPTION OF THE DRAWINGS

FIG. 1 is a cross section of a quail egg. Label 1 is the yolk, label 2 is the vitelline membrane, label 3 is the inner and outer membranes, label 4 is the nucleus, and label 5 is the albumen.

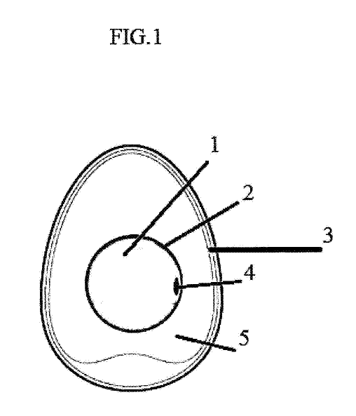

### DETAILED DESCRIPTION OF THE INVENTION

The vitelline membrane of an egg is a novel, simple and improved model of the human BBB. The vitelline membrane of an egg is very impermeable to many substances that are known to be impermeable to the human BBB. The permeability of the vitelline membrane may change after fertilization.

Although the model may utilize eggs of many living creatures such as birds, reptiles, amphibians and monotremes, the quail egg was chosen because of its size, cost and availability. Using the quail egg vitelline membrane as a model of the human BBB is not obvious from its structure or function. The cost of this model is significantly less than other models such as Caco-2, PAMPA, IAM and in silico assays. The ability to predict whether a compound will cross the BBB is extraordinary useful for drug development.

In the best embodiments of the invention non-fertilized quail eggs are decalcified in white vinegar (5% acetic acid) for 24 hours. After washing the decalcified eggs they are incubated with the proposed drug at room temperature at pH 6-8 in phosphate buffered saline (PBS) or methanol for 2 or 12 hours. The eggs are then frozen in a home freezer for 24 hours after which time a cross section of the egg is sliced with a razor knife. For testing the permeability of colored agents dissolved in PBS direct observation will show the permeability across the inner and outer membranes into the albumen and across the vitelline membrane into the yolk. None of the tested colored agents crossed the vitelline membrane. (Table 2) Observation under ultra violet light will show the permeability of fluorescent agents such as fluorescein and quinine. None of the fluorescent agents crossed the vitelline membrane. (Table 2) For those agents that are not colored but have a characteristic odor such as ethanol and diethyl ether, the odor can be detected in the albumen and yolk. Both of these agents crossed the vitelline membrane. (Table 1) The permeability of other agents that cannot be discriminated by sight, taste or smell can be assayed using spectroscopy. When agent identification uses spectroscopy these agents are dissolved in methanol and the denatured albumen, vitelline membrane and yolk can often be easily separated. Using spectroscopic analysis, acetaminophen and 2-chloroprocaine crossed the vitelline membrane. (Table 1)

### BENEFITS TO SOCIETY

It is unlikely that an in vitro model of the human BBB will be developed that will predict with 100% sensitivity and specificity the passage of a lead drug across the human BBB because the BBB is anatomically and physiologically complex and species specific. A simple, inexpensive screen using the vitelline membrane as a model of the human BBB as proposed in this invention may help speed the development of drugs that target the CNS.

### EXPERIMENTAL SECTION

Decalcified quail eggs were prepared by soaking the eggs in household white vinegar (5% acetic acid) for 24 hr. The decalcified eggs were washed with water and stored in a home refrigerator until needed for use. The decalcified eggs were incubated in solutions of agents for 2 or 12 hours. The eggs were then frozen and cut with a razor knife. The albumen and yolk were analyzed in daylight for colored agents or in darkness under ultraviolet light for fluorescent agents. Some samples of the albumen and yolk were analyzed by taste or analyzed with a spectrophotometer. Substances that were present in the yolk and had crossed the vitelline membrane are listed in Table 1.

An Ultrospect III spectrophotometer (Pharmacia, Cambridge, England) was recently calibrated by Spectrofuge Corporation of North Carolina, Inc. (Durham, N.C.). Blank quartz cuvettes were selected with the smallest deviation in absorbance. Standard solutions of 2.6×10⁻³M acetaminophen and 4.4×10⁻³ 2-cholorprocaine were prepared. Approximate peak ultraviolet wavelengths were determined with the standard solutions in methanol and PBS. Fine adjustment of the optimum wavelength was performed by retrofit of Beer's law plots of the standards and dilutions. Optimum absorbance wavelengths for acetaminophen and 2-chloroprocaine were 300 nm 329 nm, respectively. Optimum absorbance wavelengths for acetaminophen and 2-choroprocaine dissolve in PBS were 319 nm and 340, respectively. (Table 3)

The decalcified eggs were soaked in solutions for 2 or 12 hours and then placed in a home freezer prior to analysis. The quail eggs were opened with a razor knife and the denatured albumen and yolk were separated. In most samples the vitelline membrane could be easily separated from the yolk. Samples of the denatured albumen and yolk were dissolved in 5 ml of methanol or PBS and centrifuged at 2700 rpm for 5 minutes. The supernatants were then clarified through a 0.2 micrometer filter (MILLEX-FG, Merck Millipore, Ltd). Clarification of the PBS samples was more difficult than the methanol samples. The clear filtrate was placed in quartz cuvettes. Any air bubbles were eliminated and the samples were analyzed in the spectrophotometer. (Tables 4&5)

TABLE 1
Agents that cross the vitelline membrane of decalcified quail eggs

| | | | | Permeability | |
|---|---|---|---|---|---|
| | | | Incubation | (outer and | Permeability |
| | | Molecular | time | inner | (vitelline |
| Agent | Concentration | weight | (hours) | membrane) | membrane) |
| Ether | 99% | 74 | 2 & 12 | + | + |
| Ethanol | 75% | 46 | 2 & 12 | + | + |
| 2- | 4 × 10−2 M | 270 | 2 & 12 | + | + |
| Chloroprocaine | | | | | |
| in methanol | | | | | |
| Acetaminophen | 2.6 × 10⁻¹ M | 151 | 2 & 12 | + | + |
| in methanol | | | | | |
| 2- | 4 × 10⁻² M | 270 | 12 | + | + |
| Chloroprocaine | | | | | |
| in PBS | | | | | |
| Acetaminophen | 2.6 × 10⁻¹ M | 151 | 12 | + | + |
| in PBS | | | | | |

TABLE 2
Agents that do not cross the vitelline membrane of decalcified quail eggs

| | | | | Permeability | |
|---|---|---|---|---|---|
| | | | Incubation | (outer and | Permeability |
| | | Molecular | time | inner | (vitelline |
| Agent | Concentration | weight | (hours) | membrane) | membrane) |
| FD&C Blue #1 | 1% standard¹ | 793 | 2 & 12 | + | − |
| FD&C Red #40 | 1% standard² | 496 | 2 & 12 | + | − |
| and FD&C Red #3 | | | | | |
| Quinine | Standard³ | 324 | 2 & 12 | + | − |
| Fluorescein | 0.01M | 332 | 2 & 12 | + | − |
| Methyl red | 0.01M | 291 | 2 | + | − |
| Potassium | 0.01M | 158 | 2 | + | − |
| permanganate | | | | | |
| Potassium | 0.01M | 294 | 2 | + | − |
| dichromate | | | | | |
| Malachite green | 0.01M | 364 | 2 | + | − |
| Creosol red | 0.01M | 404 | 2 | + | − |

¹McCormick brand food coloring
²McCormick brand food coloring
³Canada Dry brand tonic water

### Controls (Methanol and PBS)

TABLE 3
Methanol and PBS controls

| | Wave- | | | |
|---|---|---|---|---|
| | length | Time | Albumen | Yolk |
| Agent | (nm) | (hours) | (abs) | (abs) |
| Methanol | 300 | 2 | 0.99 | 0.26 |
| Methanol | 300 | 12 | 0.52 | 0.36 |
| Methanol | 329 | 2 | 0.07 | 0.09 |
| Methanol | 329 | 12 | 0.08 | 0.26 |
| PBS | 319 | 12 | 0.87 | 0.80 |
| PBS | 340 | 12 | 0.79 | 0.55 |

TABLE 4
Absorbance values of acetaminophen
and 2-chloroprocaine in methanol

| | | Wave- | | | |
|---|---|---|---|---|---|
| Agent in | Concentration | length | Time | Albumen | Yolk |
| methanol | (m/L) | (nm) | (hours) | (abs) | (abs) |
| Acetaminophen | 2.6 × 10⁻¹ | 300 | 2 | 2.85 | 2.40 |
| Acetaminophen | 2.6 × 10⁻¹ | 300 | 12 | 2.87 | 2.56 |
| 2- | 4.4 × 10⁻² | 329 | 2 | 1.86 | 1.37 |
| Chloroprocaine | | | | | |
| 2- | 4.4 × 10⁻² | 329 | 12 | 2.52 | 1.71 |
| Chloroprocaine | | | | | |

TABLE 5
Absorbance values of acetaminophen and 2-chloroprocaine in PBS

| | | Wave- | | | |
|---|---|---|---|---|---|
| | Concentration | length | Time | Albumen | Yolk |
| Agent in PBS | (m/l) | (nm) | (hours) | (abs) | (abs) |
| Acetaminophen | 2.6 × 10⁻¹ | 319 | 12 | 0.58 | 2.87 |
| 2- | 4.4 × 10⁻² | 340 | 12 | 0.48 | 1.04 |
| chloroprocaine | | | | | |

### REFERENCES

Bellairs, R., Harkness, M., Harkness, R. D. (1963). The Vitelline Membrane of the Hen's Egg: a Chemical and Electron Microscopical Study. J. Ultrastructure Research, 8, 339-359.
Garcia, F. J., Pons, A., Alemany, M., & Palou, A. (1983). Permeability of chicken egg vitelline membrane to glucose, carbohydrate gradients between albumen and yolk. Comp Biochem Physiol B, 75(1), 137-140.
Goldberg, J. S. (2010). Selected Gamma Aminobutyric Acid (GABA) Esters may Provide Analgesia for Some Central Pain Conditions. Perspect Medicin Chem, 4, 23-31.
Goldberg, J. S. (2011). Low Molecular Weight Opioid Peptide Esters Could be Developed as a New Class of Analgesics. Perspect Medicin Chem, 5, 19-26. doi: 10.4137/PMC.S6803
Naik, P., & Cucullo, L. (2012). In vitro blood-brain barrier models: current and perspective technologies. J Pharm Sci, 101(4), 1337-1354. doi: 10.1002/jps.23022
Pajouhesh, H., & Lenz, G. R. (2005). Medicinal chemical properties of successful central nervous system drugs. NeuroRx, 2(4), 541-553. doi: 10.1602/neurorx.2.4.541
Pons, A., Garcia, F. J., Palou, A., Alemany, M. (1985). Permeability of Chicken Egg Vitelline Membrane to Amino Acids-Binding of Amino Acids to Egg Proteins. Comp. Biochem. Physiol., 82A(2), 289-292.
Wilhelm, I., & Krizbai, I. A. (2014). In vitro models of the blood-brain barrier for the study of drug delivery to the brain. Mol Pharm, 11(7), 1949-1963. doi: 10.1021/mp500046f

Having described my invention, I claim:

1. A method to predict whether or not a drug will cross the human blood brain barrier comprising incubating an egg in a solution of the drug and assessing the presence of the drug in the yolk of the egg after the drug has crossed the vitelline membrane into the yolk.

2. The method of claim 1 where the egg is from a bird, reptile, amphibian or monotreme.

3. A method to predict whether or not a drug will cross the human blood brain barrier comprising incubating a decalcified quail egg in an aqueous buffered or alcohol solution of the drug at 20-40 degrees centigrade and at a pH of 6-8 and determining presence of the drug in the yolk of the egg after the drug has crossed the vitelline membrane into the yolk.

4. The method of claim 3 where the determination of the drug is by sight or taste or smell.

5. The method of claim 3 where the determination of the drug is performed by spectroscopy.
